(19) United States
(12) Patent Application Publication
(10) Pub. No.: US 2017/0234878 A1
(43) Pub. Date: Aug. 17, 2017

(54) COMPOSITIONS FOR DETECTING CANCER CELLS ON A CELLULAR SURFACE

(71) Applicant: ISI Life Sciences, Inc., Newport Beach, CA (US)

(72) Inventors: Gerald F. Swiss, Rancho Santa Fe, CA (US); Robert M. Moriarty, Michiana Shores, MI (US)

(73) Assignee: ISI Life Sciences, Inc., Newport Beach, CA (US)

(21) Appl. No.: 15/042,103

(22) Filed: Feb. 11, 2016

(51) Int. Cl. G01N 33/574; G01N 33/58

(52) U.S. Cl. CPC G01N 33/574; G01N 33/587; G01N 33/582; G01N 2333/765

(57) ABSTRACT

Disclosed are compositions and methods for assessing the presence of tumor cells amongst normal cells.

### FIELD OF THE INVENTION

This invention provides for compositions and methods for assessing the presence of tumor cells amongst normal cells. In particular, this invention provides for albumin nanoparticles that are admixed with profluorescent moieties that are preferentially absorbed by tumor cells whereupon the profluorescent moieties are converted in situ to fluorescent moieties. Also provided are methods for using such compositions including determining the presence of tumor cells after surgical removal of tumors so as to determine if all of the tumor cells have been removed; providing solid mass tumor imaging; and assessing the efficacy of treatment protocols for solid mass tumors.

### BACKGROUND

After surgical resection of a tumor, there remains a concern that portions of the tumor, including tumor cells, remain at the tumor site. Such a concern is well justified as remaining tumor components can regenerate the underlying tumor necessitating the surgical resection.

To date, fluorescent moieties such as fluorescein have been covalently linked to albumin nanoparticles in an attempt to detect remaining tumor components. Tumor cells preferentially uptake these albumin nanoparticles because these cells overexpress SPARC which mediates albumin uptake by these cells. However, application of such compositions directly to a surgical field is deficient in that the fluorescein bound to the nanoparticles is fluorescent regardless of where it is applied. This creates a background fluorescence that renders meaningful detection of only cancer cells as opposed to normal cells very difficult.

In addition, current solid mass tumor imaging requires the use of imaging dyes or radioactive agents such as radioactive glucose. Imaging dyes are known to cause renal complications in many patients and the use of radioactive agents requires a host of safety issues for both the clinicians and patients.

Thus, there remains a need to provide for improved means for detecting cancer cells where the composition applied to surgical sites has little to no background fluorescence. Such compositions and methods would result in primarily establishing fluorescence in tumor cells. The use of UV light sources would permit the surgeon to readily assess both the quantity of remaining tumor components.

### SUMMARY

Albumin nanoparticles are known to render otherwise water insoluble hydrophobic components such as paclitaxel miscible or soluble in water. This allows for the effective delivery of such medicaments. However, such medicaments are therapeutic in nature and provide no information to the surgeon as to whether all of the tumor components have been removed or allow for tumor imaging. For example, in basal cell carcinomas, the attending clinician must often compromise between removing a sufficient amount of tissue to capture all of the carcinoma and retaining a sufficient amount of tissue so that an acceptible cosmetic appearance of the patient is retained. Likewise, for bladder tumors, it is common to conduct transurethral resection of the tumor. Once the tumor is removed, the attending clinician attempts to remove any remnants of the tumor by laser burning. However, given the transurethral aspects of this technique, it is difficult to assess the success of such lase treatment.

This invention provides for fluorescein diesters wherein the diesters are solubilized in albumin nanoparticles. As per the above, these nanoparticles are preferentially absorbed or internalized by the tumor cells. Once internalized, intracellular esterases and/or lipases remove one or both of the ester groups thereby converting the pro-fluorescent compound to a fluorescent compound resulting in rapid visualization of tumor cells. In one embodiment, the composition can be sprayed on to the surface of skin or tissue suspected of containing cancer cells. The composition will undergo preferential absorption into cancer cells (if present) and will illuminate such cells with little or no background fluorescence. It is contemplated that such illumination will occur within 1 to 30 minutes after application and will provide the clinician with an immediate evaluation of the presence of cancer cells whether for diagnostic of clinical purposes.

In another embodiment, the albumin compositions can be administered to the patient by, for example, intravenous administration. The albumin is then absorbed by the cancer cells and the tumor can be imaged by conventional techniques.

Accordingly, in one embodiment, there is provided a composition suitable for detecting cancer cells, the composition comprising a fluorescein diester solubilized in nanoparticles of albumin. These diesters are non-fluorescent and preferably comprise 5 to 50 carbon atoms wherein from 1 to 4 of the carbon atoms are optionally replaced with a heteroatom selected from oxygen, sulfur, and —NR—, wherein R is hydrogen or an alkyl group of from 1 to 20 carbon atoms. The nanoparticles preferably have an average size as measured along the longest axis of less than about 1 micron and more preferably from about 10 to 400 nanometers (nm), or 20 to about 400 nm or from about 10 to about 200 nm.

In some embodiments, the average size as measured along the longest axis is no more than about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 nm, including increments therein.

These compositions contain an amount of the fluorescein diester such that upon absorption by the tumor cells, deacylation occurs and the tumor cells will become visible under UV light. In a preferred embodiment, the compositions of this invention preferably contain from about 1:0.000001 to about 1:0.01 (w/w) albumin to the non-fluorescent fluorescein diesters. This includes ratios of about 1:0.00001, 1:0.0001, and 1:0.001 (w/w) albumin to the non-fluorescent fluorescein diesters.

Alternatively, the amount of fluorescein present in the albumin is preferably about 0.01 ppm to 10 ppm.

In another embodiment, there is provided a method for detecting the presence of cancer cells in a cellular composition suspected of containing cancer cells, the method comprising a) contacting a sufficient amount of the composition described above onto the cellular surface; b) maintaining said composition under conditions where any cancer cells present absorb the nanoparticles and deacylate at least one of the ester groups; and c) applying UV light to said surface such that any cancer cells present will fluoresce thereby self-identifying these cells as cancer cells.

In yet another embodiment, there is provided hydrophobic fluorescent diesters comprising from 5 to 50 carbon atoms (and preferably 10 to 50 carbon atoms) wherein from 1 to 4 of the carbon atoms are optionally replaced with a heteroatom selected from oxygen, sulfur, and —NR—, wherein R is hydrogen or an alkyl group of from 1 to 20 carbon atoms.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%,1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “alkyl” as used herein refers to straight chain and branched chain saturated or partially unsaturated alkyl groups having from 1 to 30 carbon atoms, and typically from 1 to 20 carbons or, in some embodiments, from 1 to 18, 1 to 15, 1 to 12, 1 to 10, 1 to 8, 1 to 6, or 1 to 4 carbon atoms. In other embodiments, the alkyl group is from 5 to 30 carbon atoms, or from 8 to 30 carbon atoms or from 8 to 20 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, 2,2-dimethylpropyl and a C₁₇ straight chain group. An example of a partially unsaturated alkyl groups are an oleyl group, and an isopropenyl group

The term “nanoparticle” as used herein refers to particles with at least one dimension less than 1 microns. In preferred embodiments, such as for intravenous administration, the nanoparticle is less than 0.1 micron.

In a population of particles, the size of individual particles is distributed about a mean. Particle sizes for the population can therefore be represented by an average, and also by percentiles. D50 is the particle size below which 50% of the particles fall. 10% of particles are smaller than the D10 value and 90% of particles are smaller than D90. Where unclear, the “average” size is equivalent to D50.

The term “profluorescent” means that a fluorescent molecule has been reversibly derivatized into a non-fluorescent molecule. Upon reversal or partial reversal of the derivatization, the resulting molecule is once again fluorescent.

### Compositions

Profluorescent fluorescein molecules are readily prepared by esterification of both hydroxyl groups. In one preferred embodiment, commercially available fluorescein is contacted with an excess of an acid anhydride under esterifying conditions. Illustrative acid anhydrides include, but are not limited to, butenoic anhydride, octanoic anhydride or oleic anhydride. Mixed anhydrides can also be employed so as to provide for a mixed fluorescein diester. These diesters can be prepared by combining equal amount of a first and second organic acid followed by dehydration. Compounds obtained by such esterification are exemplified by the following formula:

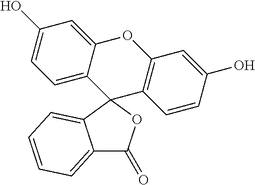 [SMILES: O=C1OC2(c3ccc(O)cc3Oc3cc(O)ccc32)c2ccccc21]

wherein each OH group is replaced by an —OC(O)R group wherein R is independently an alkyl group of from 1 to 30 carbon atoms and preferably from 5 to 30 carbon atoms and even more preferably from 8 to 30 carbon atoms.

The reaction conditions for esterification are exceptionally well known in the art and do not form part of this invention. However, the resulting diesters comprise part of this invention to the extent that such diesters are novel.

A suitable amount of a profluorescent fluorescein diester is admixed with albumin in sufficient amounts such that upon deacylation, the fluorescence arising from the resulting fluorescein monoester or fluorescein can be detected. As fluorescein is highly fluorescent, it is contemplated that the amount of the pro-fluorescent compound combined with the albumin can be as little as 0.01 ppm, 10 ppm, or 100 ppm, or 1000 ppm based on 1,000,000 parts of albumin on a weight to weight basis. Preferably, the weight ratio of the profluorescent compound to albumin used in the invention described herein ranges from 1:100,000 or 1:10,000 or 1:1000 or 1:100.

Albumin may comprise bovine serum albumin, human serum albumin, or a combination thereof. Albumin (e.g., bovine serum albumin and human serum albumin) is commercially available and methods to provide nanoparticles of the profluorescent fluorescein moiety in albumin are known in the art and can follow the protocols provided in any of U.S. Pat. Nos. 7,758,891; 7,820,788; 7,923,536; 8,034,375; 8,138,229; 8,268,348; 8,314,156; 8,853,260; and 9,101,543, each of which is incorporated herein by reference in its entirety.

Accordingly, in one aspect, disclosed herein are compositions suitable for detecting cancer cells at a surgical site, the composition comprising a fluorescein diester solubilized in nanoparticles of albumin wherein said fluorescein diesters are non-fluorescent. In some embodiments, said nanoparticles have an average size as measured along the longest axis of from about 100 to about 400 nanometers. In some embodiments, each ester group of said fluorescein diester has from 1 to 30 carbon atoms provided that at least one of said ester groups has from 5 to 30 carbon atoms. In some embodiments, each ester group of said fluorescein diester has from 5 to 30 carbon atoms. In order to avoid any ambiguity, the number of carbon atoms in the ester group includes the carbon atom of the ester functionality [—OC(O)]—. In some embodiments, said fluorescein diester is selected from the group consisting of fluorescein dibutyrate, fluorescein dioctate, and fluorescein dioleate.

In another aspect, disclosed herein are compositions suitable for detecting cancer cells at a surgical site, the composition comprising a fluorescein diester solubilized in nanoparticles of albumin wherein said fluorescein diesters are non-fluorescent; each ester group of said fluorescein diester has from 1 to 30 carbon atoms provided that at least one of said ester groups has from 5 to 30 carbon atoms; and said nanoparticles have an average size as measured along the longest axis of from about 10 to about 400 nanometers.

In another aspect, disclosed herein are compositions suitable for detecting cancer cells at a surgical site, the composition consisting essentially of a fluorescein diester solubilized in nanoparticles of albumin wherein said fluorescein diesters are non-fluorescent; each ester group of said fluorescein diester has from 1 to 30 carbon atoms provided that at least one of said ester groups has from 5 to 30 carbon atoms; and said nanoparticles have an average size as measured along the longest axis of from about 10 to about 400 nanometers.

### Methods

These nanoparticles can then be applied to a cellular surface suspected of containing cancer cells. In one embodiment, a sterile aqueous mixture (e.g., dispersion or solution) of these nanoparticles is “painted” onto such a cellular surface including a surgical surface after removal of a solid mass tumor. In another embodiment, a sterile aqueous mixture (e.g., dispersion or solution) of these nanoparticles is “sprayed” onto a cellular surface suspected of containing cancer cells. In either case, the nanoparticles are then preferentially absorbed by the tumor cells. Intracellular enzymes (e.g., esterases and lipases) will then deacylate at least one of the ester groups and preferably both ester groups. Regardless of whether there is deacylation that removes one or both of the esters, the deacylated fluorescein compounds now fluoresce. Such provides an immediate (e.g., 5 to 30 minute) response time that allows the surgeon to survey the surgical field with UV light to assess the presence of fluorescence which, as above, indicates the presence of tumor cells.

Of course, the presence of fluorescence will permit the surgeon to remove remaining remnants of the excised tumor thereby providing the patient with a significant benefit in terms of increased likelihood of recovery.

Accordingly, in one aspect, disclosed herein are methods for detecting the presence of cancer cells in a cellular surface suspected of containing cancer cells, the method comprising a) contacting a sufficient amount of the albumin composition described above onto the cellular surface; b) maintaining said composition under conditions where any cancer cells present absorb the nanoparticles and deacylate at least one of the ester groups; and c) applying UV light to said surface such that any cancer cells present will fluoresce thereby self-identifying these cells as cancer cells. In one embodiment, the cellular surface is a surgical field after surgical resection of a tumor. In another embodiment, the surgical field comprises basal cells suspected of containing basal cell carcinoma. In another embodiment, the surgical field comprises a mole suspected of being a melanoma tumor and the mole is surface is cut to a level when the albumin composition can be applied and a determination of whether the mole contains cancer cells can be evaluated by UV light. In another embodiment, the surgical field includes bladder tissue after tumor resection. In another embodiment, the surgical field includes a tissue sample removed from the patient and immediately assessed to determine the presence of cancer cells.

In another aspect, there is provided a method for the systemic uptake of the albumin compositions described herein wherein the compositions are administered to a patient in an amount sufficient to be absorbed by cells of the tumor(s) present. Application of UV light will identify the presence of such tumors and permit imaging of the tumor size and shape. In another embodiment, such a diagnostic evaluation of the tumor can be used to assess the efficacy of the therapeutic treatment protocol used to treat the tumor.

In another aspect, disclosed herein are methods of treating cancer in a patient in need thereof, the method comprising comprising a) contacting a sufficient amount of the albumin composition described above onto the cellular surface; b) maintaining said composition under conditions where any cancer cells present absorb the nanoparticles and deacylate at least one of the ester groups; and c) applying UV light to said surface such that any cancer cells present will fluoresce thereby self-identifying these cells as cancer cells; and (d) removing the cancer cells.

In another aspect, disclosed herein are methods of treating cancer in a patient in need thereof, the method comprising removing from the patient one or more cancer cells detected using a method of detecting the presence of cancer cells disclosed herein.

### EXAMPLES

### Example 1

### Preparation of Dibutyl Fluorescein Diester

5 grams of fluorescein is dissolved in pyridine. 2.1 equivalents of butanoic anhydride are added thereto. The reaction mixture is maintained at 40° C. for a period of time sufficient to substantially complete the reaction. The resulting diester is recovered by conventional means. Alternatively, fluorescein dibutyrate can be obtained commercially from, for example, Chemodex Ltd., Lidenstrasse 77, 900 St. Gallen, Switzerland.

### Example 2

### Preparation of Dioleyl Fluorescein Diester

5 grams of fluorescein is dissolved in pyridine. 2.1 equivalents of commercially available oleic anhydride are added thereto. The reaction mixture is maintained at 40° C. for a period of time sufficient to substantially complete the reaction. The resulting diester is recovered by conventional means.

### Example 3

### Albumin Example

0.3 milligrams of the dioleyl fluorescein diester of Example 2 are dissolved in 3 mL of methylene chloride. The resulting solution is added to 270 mL of an aqueous solution containing 1% human serum albumin (HSA). The mixture is first homogenized at low RPM to form a crude emulsion and then transferred to a high pressure homogenizer where a homogenous emulsion is formed. The resulting emulsion is then placed in a rotary evaporator so as to remove the methylene chloride. The resulting dispersion contains about 1 ppm of the diolelyl fluorescein diester.

### Example 4

### Application of the Dispersion onto a Mixed Cell Population

1 mg of the albumin dispersion of Example 3 is admixed with 10 mL of distilled water. The resulting admixture is placed into a spray device such that a fine mist of the dispersion can be generated.

A tumor on the flank of a mouse is surgically excised. After removal, the clinician applies the albumin dispersion over the surgical field using the spray device as described above. The applied nanoparticles are preferentially absorbed by the tumor cells whereupon cellular enzymes deacylate one or both of the ester groups so as to convert the non-fluorescent diester to a fluorescent derivative. Application of UV light to the surgical field will provide fluorescence for those tumor cells which remain.

1. A composition suitable for detecting cancer cells, the composition comprising a fluorescein diester solubilized in nanoparticles of albumin.

2. The composition of claim 1 wherein each of said esters independently comprise 5 to 50 carbon atoms wherein from 1 to 4 of the carbon atoms are optionally replaced with a heteroatom selected from oxygen, sulfur, and —NR—, wherein R is hydrogen or an alkyl group of from 1 to 20 carbon atoms.

3. The composition of claim 1 wherein said nanoparticles have an average size as measured along the longest axis of less than about 1 micron.

4. The composition of claim 3, wherein said nanoparticles have an average size for from about 10 to 400 nanometers.

5. The composition of claim 3, wherein said nanoparticles have an average size of from about 20 to about 400 nanometers.

6. The composition of claim 3, wherein said nanoparticles have an average size of from about 10 to about 200 nm.
